(19) United States
(12) Patent Application Publication
(10) Pub. No.: US 2022/0395631 A1
(43) Pub. Date: Dec. 15, 2022

(54) RADIALLY ADJUSTABLE MULTI-CARTRIDGE COMBINATORIAL DRUG DELIVERY DEVICE FOR SUBCUTANEOUS INJECTION

(71) Applicant: Bristol-Myers Squibb Company, Princeton, NJ (US)

(72) Inventors: Martin John McLoughlin, Hillsborough, NJ (US); Stephen Lawrence Zieminski, East Brunswick, NJ (US); Peter William Heyman, Florham Park, NJ (US); Mark Steven Howansky, Green Brook, NJ (US); Frank Berardocco, Old Bridge, NJ (US); Samuel Robert Zschack, Palatine, IL (US); Melanie Marie Springer, Baltimore, MD (US); Jonathan Adam Reed, Baltimore, MD (US); Joseph Morin, Riverton, UT (US)

(21) Appl. No.: 17/771,935

(22) PCT Filed: Nov. 9, 2020
(86) PCT No.: PCT/US2020/059672

§ 371 Date: Apr. 26, 2022

Related U.S. Application Data
(60) Provisional application No. 62/932,968, filed on Nov. 8, 2019.

(51) Int. Cl. A61M 5/00; A61M 5/14; A61M 5/145; A61M 5/142

(52) U.S. Cl. CPC A61M 5/002; A61M 5/1408; A61M 5/1454; A61M 5/14244; A61M 2005/005

(57) ABSTRACT

In one aspect, a drug delivery device is provided for delivering drug from a plurality of drug cartridges. The drug delivery device includes: a cylindrical cassette configured to accommodate the plurality of drug cartridges; a reversibly advanceable plunger; and, an indexer for incrementally rotating the cassette to align the plurality of drug cartridges individually with the plunger. The indexer includes first and second shafts with cooperating elements which cause incremental rotation of the first shaft, relative to the second shaft, upon the second shaft engaging the first shaft.

### CROSS-REFERENCE TO RELATED APPLICATIONS

This application is a National Stage Application under 35 U.S.C. § 371 of PCT Application No. PCT/US2020/059672, filed Nov. 9, 2020, which claims the priority benefit of U.S. Provisional Application No. 62/932,968, filed Nov. 8, 2019; the contents of which are herein incorporated by reference in their entireties.

### FIELD OF THE INVENTION

The field of the present invention is the administration of liquid drugs subcutaneously. More particularly the invention relates to the subcutaneous administration of combinations of two or more liquid drugs in fixed ratios by weight.

### BACKGROUND TO THE INVENTION

For a variety of reasons, many drugs must be administered parenterally. For example, biotechnology derived biologic drugs are therapeutic proteins which cannot be delivered orally because they would be destroyed by the digestive system, rendering them ineffective. Therefore, such biologic drugs are typically delivered via routes of administration that bypass the digestive system, most typically via the intra-venous and sub-cutaneous routes.

Recent advances in medicine, particularly in the treatment of cancer, have demonstrated that therapeutically beneficial effects can be achieved by the synergistic combination of two or more drugs.

For example, recent clinical research has demonstrated that the combination of an anti-PD-1 checkpoint inhibitor drug with a CTLA4 checkpoint inhibitor can have beneficial synergistic effects in some tumor types, which can lead to better clinical outcomes than could be achieved by the individual administration of either drug alone. Such checkpoint inhibitor drugs are often biotechnology derived monoclonal antibodies or fragments thereof of the immunoglobulin type. In some situations, it may be beneficial to combine such biologic drugs with conventional chemotherapy agents such as cytotoxic drugs.

Co-administration of drugs parenterally presents several challenges and various approaches have been used to overcome them. These challenges include the increased medication complexity, the greater risk of medication errors, and the burden on the patient. Greater medication complexity can manifest in several ways depending on how the drugs are combined and administered. For example, one approach to combining therapeutic biologic drugs has been through co-formulation into fixed ratio combinations in a solution. This results in formulation complexity because of the need to ensure a stable formulation in which the combined drugs maintain their potency and quality throughout the pharmaceutical supply chain. The person of ordinary skill in the art will appreciate that such drug formulations will typically include several excipients for example buffers, pH modifiers, tonicity modifiers, stabilizers and so on. As the number of drugs in combination increases, the formulation complexity grows. Associated with the formulation challenges are challenges related to the development of analytical methods for such complex formulations such as assays to evaluate the quality, potency and strength of each drug in the mixture. A further limitation of the fixed ratio combination is that flexibility in the ratios of the drugs to be administered is lost.

Formulation and analytical complexity can be avoided and dosing flexibility maintained by compounding the drugs together from single-agent formulations closer to the point of care, for example in a compounding pharmacy. In this case, the pharmacist or pharmacy technician follows a protocol for the mixing of the separate drugs using aseptic technique under a pharmacy hood. Most typically, this approach is currently applied to the mixing of drugs in an intra-venous infusion bag though the method could in principle be applied to the mixing into a vial for subsequent sub-cutaneous injection. Although formulation and analytical complexity is avoided by this approach, the complexity is transferred to the pharmacy. When this method is used to prepare intra-venous infusions, it can only be performed close to the point of care for patients attending infusion clinics. The advantage in dose and dose ratio flexibility provided by this approach creates an attendant risk of medication errors in the pharmacy e.g. by use of the incorrect drugs or mixing them in incorrect ratios. The checks and controls used in a well-organized pharmacy are designed to prevent such medication errors but this risk provides another reason why this practice is restricted to pharmacies close to the point of care such as within the hospital. A final risk with pharmacy compounding is the risk of exposure to the drugs or needle-stick injuries as a result of the multiple needle-based transfers that must be performed. This risk may be reduced by the use of compounding machines in pharmacies however such machines become another source of complexity and expense.

Formulation and analytical complexity can also be avoided by administering the drugs separately, e.g. in separate intra-venous infusions or sub-cutaneous injections. In some circumstances, this approach may be necessary for technical reasons, for example, if a stable fixed ratio formulation cannot be achieved. In the intra-venous case, this approach provides only a marginal reduction in protocol complexity for the pharmacy which now needs to manage multiple compounded infusions. This approach also does not eliminate the risk for medication errors. In both the intra-venous infusion and sub-cutaneous injection cases the burden on the patient is greater because they must now endure multiple infusions or injections.

In some circumstances, for reasons of safety, it may not be possible to administer all of the drugs at once. For example, the excipient burden may be unacceptably high. In the case of biologic drugs derived from bacterial cell cultures, the residual bacterial endotoxin concentration, though controlled to as low a level as possible in downstream processing, may nonetheless prevent the at once administration of multiple drugs in a combination. The need to manage the excipient and endotoxin burden may require that the patient remains at the hospital for a period of days or must attend the clinic over several days, further adding to the burden on the patient.

In principle, drugs intended for co-administration could be provided separately in convenient pre-filled presentations for sub-cutaneous delivery such as pre-filled syringes, auto-injectors or body-worn injectors and self-administered by the patient individually away from the clinical setting. This approach could alleviate the need for the patient to remain at the hospital or make multiple visits, however such an approach would result in multiple injections and hence patient inconvenience and the other attendant safety risks such as injection site reactions. This approach also creates a significant risk of medication error as patients must keep track of their administration status for each drug in the combination. If for safety or therapeutic reasons the timing of administration of the respective drugs is important, e.g. to manage endotoxin limits, then the risk for medication errors due to incorrect timing of constituent doses also arises. Medication error risks could be mitigated to some extent by co-packaging along with clear instructions but cannot be eliminated entirely.

Currently, for the aforementioned reasons, most parenterally administered drug combinations are administered by the intra-venous route in clinical environments.

For pharmaceutical companies that manufacture and distribute drugs as combination therapies, the co-formulation approach creates additional challenges and complexity in manufacturing and the supply chain. For companies with portfolios of individual drugs used in combination with each other these complexities increase as the number of combinations offered increases.

Each new combination of drugs adds additional single-keeping units (SKUs) to finished goods inventories. Furthermore, each new ratio or strength of drugs adds still more SKUs. Such rapid growth of SKUs is known in the discipline of supply chain management as ‘combinatorial explosion’. In accounting terms, stocks of such SKUs are accounted for as finished goods inventory. Additional complexities arise in work-in-process (WIP) inventories because the individual drug substances or active pharmaceutical ingredients (APIs) must be stored in bulk until compounding. Subsequently the bulk compounded drug product must likewise be stored until filling into unit doses. In the case of biologic drugs which are typically stored deep-frozen, this results in multiple freeze-thaw processes and the associated need for large refrigerated storage facilities and equipment.

Particularly when the drugs in question are costly biologic drugs, the financial impact of combinatorial explosion and associated inventory growth can be significant not only because of the working capital tied up as inventory but also because of the expensive facilities required to store WIP and finished goods inventory under refrigerated conditions.

A further challenge with co-formulated drug combinations arises in supply chain planning and forecasting due to the challenge of optimizing the product mix amongst the various possible combination SKUs in response to market demands. Because the bulk stored APIs are ‘fragmented’ amongst a potentially large number of finished goods SKUs, accurate forecasting of demand is vital to minimize the risk of overstocking in some SKUs and understocking (‘stock-outs’) in others. With costly biologic drugs the costs associated with forecasting errors can be very large. This issue is further exacerbated because pharmaceuticals are perishable goods meaning that unsold inventory can only be stored for a fixed period before they are written-off. Clearly, such forecasting challenges grow with the number of drugs used in combinations and SKUs in the product portfolio.

In summary, for all of the aforementioned reasons, there is a need for technologies that can address each of these challenges associated with the delivery of combination therapies. The ideal technology would avoid the formulation and analytical complexities of fixed ratio combination formulations, avoid combinatorial explosion and inventory growth in manufacturing and the supply chain, eliminate the risk of medication errors in pharmacies and at the point-of-care (whether in-clinic or at-home) and minimize patient burden associated with multiple infusions or injections and dose-timing restrictions. The ideal technology should also maximize patient convenience by enabling the flexible and convenient delivery of combination therapies, for example in the home or other non-clinical environments. To maximize patient convenience, the ideal technology should enable sub-cutaneous administration, as this is more suitable for non-clinical environments. It should also anticipate advances in medicine such as the development of more complex combination therapies comprising three or more drugs and active excipients such as hyaluronidase enzyme (for example, recombinant human hyaluronidase enzyme, marketed under the brand name ENHANZE® by Halozyme Therapeutics Inc, San Diego Calif.).

As a further example of medical advances, recent advances in the science of immuno-oncology suggest that there may be therapeutic benefits to the precision timing of the constituent drug doses of combination therapies. For example, consider a combination therapy of ‘Drug A’ and ‘Drug B’ designed to respectively target two biochemical targets ‘A’ and ‘B’ expressed by a particular tumor type. Recent developments suggest that in some cases there may be a temporal aspect to the expression of the targets by the tumor which can be influenced by the timing of the administration of the respective drugs. For example, administration of Drug A to bind with target A at time zero, may stimulate or up-regulate the expression of target B sometime after, which may be minutes, hours, or even days. In such a case, it may be optimal to administer Drug B when the peak expression of target B occurs. Such temporally resolved dosing may be optimal for reasons of safety (for example reducing the required drug dose for equivalent therapeutic effect), efficacy or both.

Given the biological nature of such time resolved effects, they may be incompatible with conventional clinical schedules and so to be utilized would require visits to the clinic for infusion on unconventional schedules, increasing both the clinical and patient burden of treatment. Therefore, in order to fully exploit these effects, sub-cutaneous delivery in non-clinical settings is required to maximize the flexibility in dose-timing.

The above-described ideal technology which enables sub-cutaneous infusion is therefore also suited to temporally resolved dosing of therapeutic combinations.

Applicant has now realized that the combinatorial principles described in applicant's applications U.S. Provisional Patent Appl. No. 62/670,266, PCT Appl. No. PCT/US2019/031727, PCT Appl. No. PCT/US2019/031762, and, PCT Appl. No. PCT/US2019/031791, which are incorporated by reference herein in their respective entireties, when implemented in sub-cutaneous delivery devices can realize the requirements of the above described ideal technology.

### SUMMARY OF THE INVENTION

In a first aspect of the present invention, there is provided a cassette housing made in two-parts of broadly cylindrical shape defining a plurality of cylindrical chambers radially disposed around the circumference of said cylinder, the axes of each chamber being parallel to the axis of the cassette housing, for the receipt of liquid drug-filled cartridges. Said cassette housing further comprises a central orifice parallel to the cassette axis for the receipt of the central spindle of a drive unit. The first part of said cassette housing defines the distal end of said cartridge chambers. The distal end of the first part comprises circular apertures centered on the axis of each chamber to provide access for a plunger rod of a drive unit to the distal end of the installed cartridges. The radius of said apertures are smaller than the chamber diameter, approximating the inner-diameter of the cartridges to be installed in the chamber such that the aperture also provides a load bearing surface for the distal end of the cartridge when force is applied to the cartridge rearwardly. In embodiments, the exterior shape of the cassette housing may deviate locally from the cylindrical to define an eccentricity that may be used for alignment purposes when installed in a drive unit, provided that the chambers remain radially disposed on a circle centered on the central axis of the cassette housing.

The second part of the cassette housing defines the proximal end of each cartridge chamber and has shape and dimensions designed to fit to the first part in intimate locking engagement to form a cassette housing assembly. Hence any eccentricity defined by the first part is reflected by the design of the second part. The proximal end of the second part comprises circular apertures at the proximal end of each cartridge chamber of diameter smaller than that of the outer diameter of the crimp-seal of the cartridge to be installed but larger than the septum of the cartridge such as to define a bearing surface for the purpose of retaining the cartridges within said chamber when a force is applied forwardly to the cassette whilst also exposing the rubber septum of each drug-filled cartridge to enable access from the exterior of the cassette housing.

There is further provided a cassette cap (also referred to herein as “cassette manifold top” and “manifold top”) comprising a plurality of hollow needle tips equal in number to the chambers of the cassette housing and radially disposed on a circle centered at the center of the cassette cap and of the same radius of the corresponding circle on which the chambers of the cassette housing are disposed. Said cassette cap is shaped to mate closely with the cassette housing with mating features designed to mate in locking engagement with corresponding mating features on the cassette housing. In embodiments where an eccentricity is built into the cassette housing, this feature will be likewise present on the cassette cap to ensure mating correspondence. Each hollow needle tip of the cassette cap is angularly distributed around said circle on the same pitch as the cartridge chambers of the cassette housing such that the hollow needle tips are in mating alignment with the central axis of each chamber, and hence the installed cartridge axis when the cassette cap is in locking engagement with the cassette housing.

The cassette housing and cassette cap are dimensioned such that when in locking engagement, each hollow needle tip pierces the septum of each cartridge installed in its corresponding chamber of the cassette housing.

Each of said hollow needle tips is in fluidic communication with a fluidic channel within the body of the cassette cap. Each of said fluidic channels are combined at a single outlet port located centrally at the axis of the cassette housing assembly to which flexible tubing is attached. Hence, the hollow needle tips, fluidic channels and outlet port collectively define a fluidic manifold from each cartridge to the single outlet port and tubing. In embodiments, uni-directional check valves may be incorporated into the fluidic channels to prevent rearward flow of fluid.

To the proximal end of the tubing is connected a cannula arranged for the sub-cutaneous delivery of the cartridge contents to the patients. In embodiments said tubing may incorporate a flexible coupling to enable the rotation of the cassette housing independently of the proximal end of the tubing, thereby preventing rotation and kinking of the tubing.

In embodiments, a two-chambered cartridge arrangement could be utilized which would enable this device to be used for the reconstitution and delivery of drug product.

In embodiments, the cassette housing assembly may incorporate identification technologies including but not limited to technologies such as radio-frequency identification (RFID), near field communication (NFC) optical bar-codes and quick response codes for reading by a drive unit for purposes of identification.

In a second aspect of the present invention there is provided a drive unit defining a chamber for receipt of a cassette housing assembly according to the aforementioned description comprising a central alignment spindle for receipt by the central orifice of the cassette housing assembly, indexing means for the precise incremental rotation of said cassette housing assembly, plunger drive means for the displacement of the installed cartridge stoppers to provide forward motive force for the expulsion of the liquid drug stored within the cartridges through the fluidic manifold to the needle cannula, a source of electrical power and control means.

The drive unit further comprises a hinged, latching door for the retention of the cassette housing within the chamber, exterior user controls and indicators and means for wearable attachment to a patient. Said door defines an aperture defining a path for the flexible tubing from the interior of the drive unit to the patient.

In embodiments said drive unit may further comprise wireless communication means implementing wireless communication protocols including but not limited to personal area network transceivers (e.g. Bluetooth, BTLE, Zigbee), wireless network protocols (e.g. IEEE 802.11x also known as Wifi), cellular communications protocols (e.g. GSM, EDGE, 4G LTE). Said communications protocols may in embodiments be used to communicate data including but not limited to date, time, location of use, identity and serial numbers of drug cassettes etc.

This device could be used in a hospital or clinical setting; however, it is envisioned to be used by a patient in a home use environment. Due to the utilization of discrete cartridges, the possibility of sequential delivery of combinatorial therapies is possible as well as time-resolved dosing e.g. for therapeutic or safety reasons. In the future biomarker based dosing could be possible as patient diagnosis times and supply chain agility continues to improve.

### BRIEF DESCRIPTION OF DRAWINGS

FIG. 1—view of a standard 1.5 mL cartridge

FIG. 2—exploded view of the Cassette

FIG. 3—a complete Ready-for-Use (RTU) Cassette

FIG. 4—Manifold top and transparent view

FIG. 5—view of cassette with manifold top attached

FIG. 6—Belt worn Drive Unit

FIG. 7—View of Drive Unit UI and outer features

FIG. 8—Cassette door button with interlock

FIG. 9—Cassette revolving mechanism within drive unit

FIG. 10—Cassette revolving mechanism components

FIG. 11—Revolving mechanism operation

FIG. 12—Cross section view of Drive Unit components

FIG. 13—Plunger rod expelling cartridge contents

FIGS. 14-17—Alternate Drive Unit configuration

### DETAILED DESCRIPTION

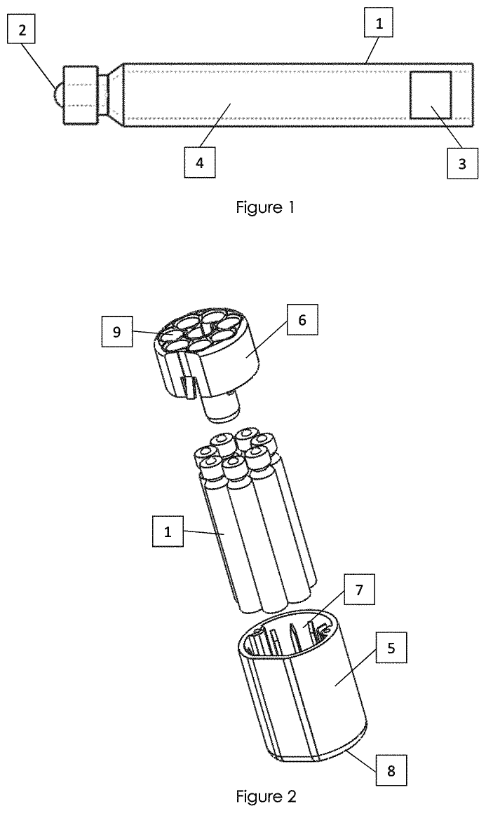

With reference to the Figures, drug combinations will be delivered and configured through the utilization of a disposable cassette 10, which contains an arrangement of liquid drug filled cartridges 1 for sequential injection. As known in the art, one or more of the cartridges may be dry/wet cartridges having separated dry and wet components allowing for solubilized, or other powder form drug, to be reconstituted by a diluent, within the cartridge (e.g., under movement of the stopper 3). As shown in FIG. 1, cartridges 1 are cylindrical glass tubes with one end being formed to accommodate a crimped septum seal 2. Once the septum seal 2 is crimped onto the cartridge 1, the cartridge 1 is filled with liquid drug product and a stopper 3 is inserted into the second open end to seal its contents. In order to dispense the fluid within the cartridge 1, a cannula 13, must first pierce the septum 2 to access the drug fluid chamber 4. With the fluidic pathway open, a force is applied against the stopper 3 compressing the fluid held inside the cartridge 1 pushing it out from the cartridge 1 through the fluid pathway of the cannula 13 transecting the septum 2.

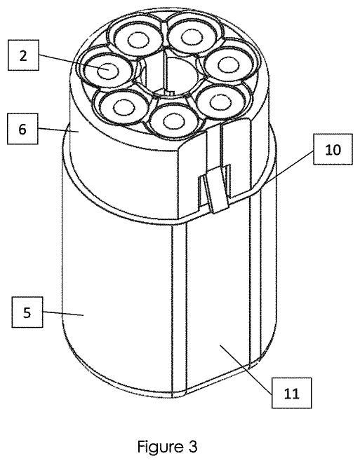

As shown in FIG. 2, the cassette 10 is used to house and load a preconfigured arrangement of cartridges 1 into the drive unit 20 for delivery to the patient. The cassette 10 consists of a main body housing 5, with holding chambers 7 for multiple cartridges 1 positioned radially around its lateral axis, and, a housing top 6, which closes over the cartridges 1 held in the main body housing 5 capturing them within the main body housing 5. Cutouts on the bottom of the main body housing 8 under each cartridge 1 allow physical access to the stoppers 3 within cartridges 1, while cutouts 9 in the housing top 6 allow open access to the cartridge septa 2 shown in FIG. 3. The cassette 10 is cylindrical in shape and is depicted with a flat 11 on its outer surface which is used to control its orientation when loaded into the drive unit 20. This unique shape is used as a keying feature and can take the form of different shapes or features in other embodiments. The cassette 10 depicted in the provided figures, demonstrates the use of seven discrete cartridges 1; if fewer cartridges are needed, less could be assembled into the cassette 10 leaving empty holding chambers 7. In embodiments where more cartridges would be needed the cassette 10 could be designed to hold additional cartridges without limit. An RFID label or equivalent technology, containing drug content and order information could be attached to the main body housing 5 to communicate with the drive unit 20 prior to delivery to ensure that an authentic and correct cassette 10 is being used.

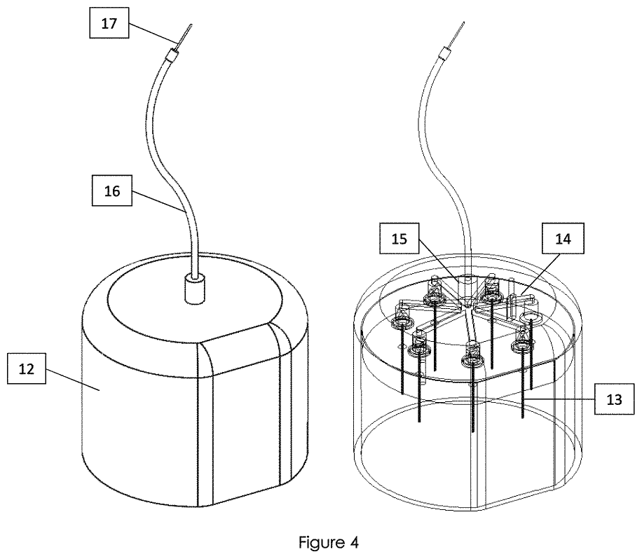

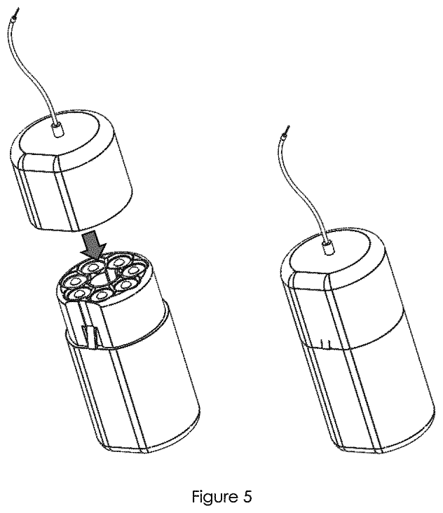

Illustrated in FIG. 4, the cassette manifold top 12, which is a body having an inner cavity negative to that of the cassette-housing top 6, is designed to be installed over the cassette housing top 6, over the cartridge septa 2, and to permanently lock to the cassette 10 body. Shown in FIG. 4, within the manifold top 12 positioned above each cartridge 1 is a sharpened cannula 13, which connects to a fluidic channel 14 within the manifold top 12, which all converge to a common output 15 at the axis of the manifold top 12. This common output 15 leads to an infusion set 16 with a needle 17 that is to be inserted into a patient's injection site on the abdomen. When the manifold top 12 is installed onto the loaded cassette 10 (FIG. 5) each cannula 13 pierces its respective septum 2 and creates a fluid path from all cartridges 1 in the cassette 10 to the infusion set 16. In embodiments, check valves could be installed inline of each cannula 13 in order to remedy back flushing into other cartridges 1 during injections. During the manufacturing process, the cannula 13 with the manifold top 12 would be hermetically sealed and the entire part with infusion set 16 would undergo a terminal sterilization process for example by use of gamma irradiation or ethylene oxide (EO).

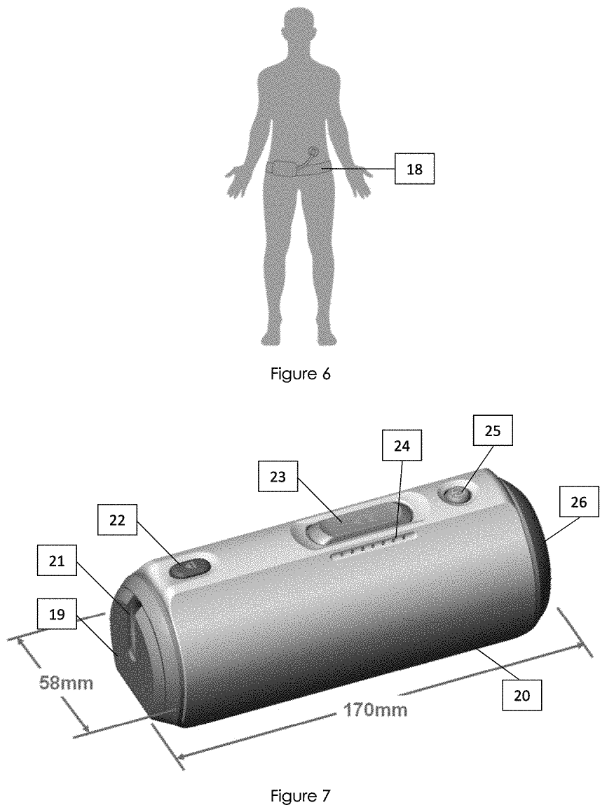

As the drug product is held within the cassette 10, it is delivered to the patient by means of an electro-mechanical belt worn drive unit 20. As shown in FIG. 6, the drive unit 20 is attached to the patient by means of a belt or body strap 18; the cassette 10 is then loaded into the drive unit 20 with the infusion set 16 freely exiting from the drive unit 20. The infusion set 16 terminates with a 25 G or similar needle 17, which is inserted into the patient's abdominal injection site.

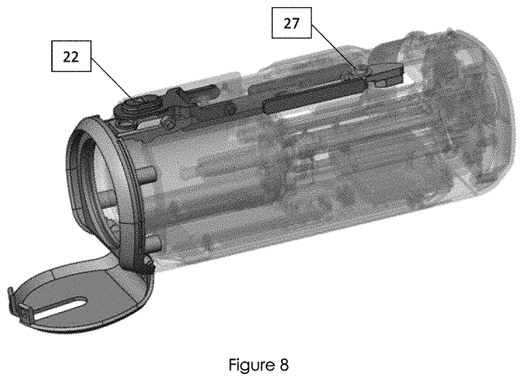

An overview of the drive unit's 20 outer features and controls are depicted in FIG. 7. On the front face of the drive unit 20 exists the cassette door 19 which is spring biased to automatically open and is used to cover the cassette receptacle drum 28 within the drive unit 20. The cassette door 19 has a cutout 21 to allow the infusion set 16 of the cassette 10 to pass through the cassette door 19 once it is closed. Atop the drive unit 20 exists a mechanical button 22, which is pressed by the user to unlatch the cassette door 19 on the front face of the device to allow it to open. To prevent users from opening the cassette door 19 during operation, the cassette door button 22 can be disabled internally via mechanical interlock 27 by the device (FIG. 8). Additionally atop the drive unit exists a simple user interface (FIG. 7) consisting of a power button 25, a start/pause button 23, and a series of progress LEDs 24. The power button 25 is pressed by the user to energize or turn off the device, while the start/pause button 23 is pressed by the user to begin or pause the infusion process. The number of LEDs 24 present on the UI is representative of the number of cartridges 1 loaded into the device. As the device progresses through the infusion process, the LEDs 24 will light up to signify the cartridge 1 has finished its infusion. These controls and indicators on the top lateral face are currently contained upon a PCB mounted behind the drive unit's 20 outer shell. In embodiments these controls could be replaced with a touch display or controlled remotely through a technology such as Bluetooth. In embodiments the individual PCB mounted LEDs may be replaced by a single organic LED (oLED) display. On the rear face of the device is a USB C connector 26, which is used as a receptacle to connect a charger to recharge the device's internal battery 39.

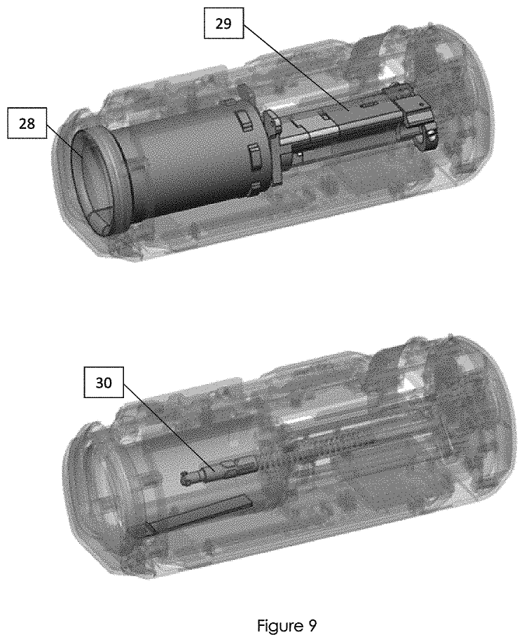

The cassette 10 is loaded into the cassette drum 28, which is shaped to accept the cassette's 10 outer shape in order to control the orientation of the cassette 10 when loaded into the drive unit 20 (FIG. 9). In the center axis of the cassette drum 28 is a spring-loaded retainer 30 which inserts into the center axis of the cassette 10. When the cassette 10 is pressed into the mechanism spring loaded tabs expand out from the retainer 30, capturing the cassette 10, locking it by its lateral axis within the cassette drum 28. The cassette drum 28 and retainer 30 are attached to the rotating end of the revolver mechanism 29. In embodiments, an RFID transmitter/receiver could be placed near the cassette drum 28 in order to identify and communicate with the loaded cassette 10.

The revolver mechanism 29, also referable to as an indexer, is configured to incrementally rotate the cassette 28, particularly to individually align the cartridge 1 with plunger rod 42.

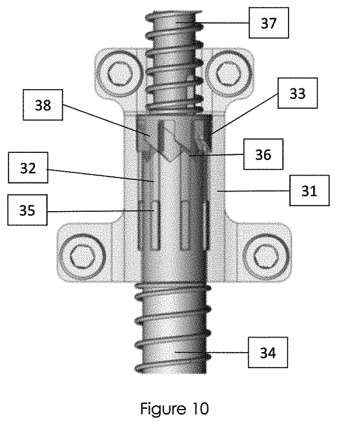

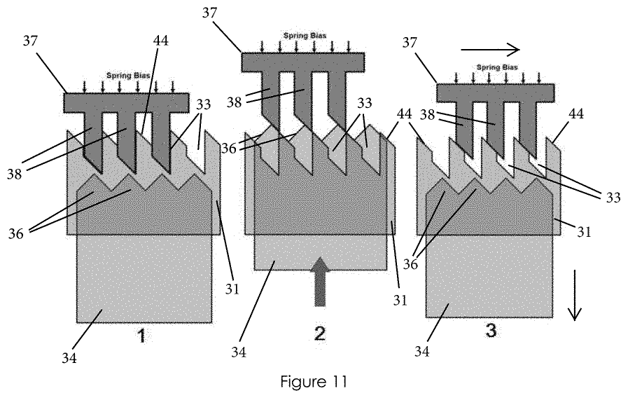

As shown in FIG. 10, the revolver mechanism 29 is comprised of a cylindrical carrier 31 which has splines 32 and notches 33 radially cut into its inner walls. Extending into the carrier 31 is a driving shaft 34, which terminates within the carrier 31. This terminal end of the driving shaft 34 is hollow with an edge shaped into teeth 36. The outer surface of the driving shaft 34 within the carrier 31 is splined 35 to interface with the splines 32 of the carrier 31 allowing the driving shaft 34 to only move vertically within the carrier 31. The driving shaft 34 is spring biased to push away from the carrier 31. The cassette shaft 37 has the cassette drum 28 and retaining mechanism 30 at one end with the cassette shaft 37 terminating within the carrier 31. This terminal end of the cassette shaft 37 ending within the carrier 31 has a ring of slant-cut teeth 38 set around the shaft that interface with the notches 33 within the carrier 31, the tip is shaped to protrude into the hollowed end of the driving shaft 34. The cassette shaft 37 is able to rotate within the carrier 31 and is spring biased towards the carrier 31. Demonstrated in FIG. 11, with the slant-cut teeth 38 of the cassette shaft 37 interfaced within the notches 33 of the carrier 31, an external force overcomes the spring bias of the driving shaft 34, pushing it into the carrier 31 making contact with the cassette shaft 37. The teeth 36 at the end of the driving shaft 34 push the cassette shaft 37 up from the carrier 31 so that the slant-cut teeth 38 of the cassette shaft 37 clear the carrier notches 33. The teeth 36 of the driving shaft 34 then act as a ramp to guide the slant-cut teeth 38 of the cassette shaft 37 into the next notch 33 of the carrier 31. As the driving shaft 34 retracts back to its starting position, the spring bias of the cassette shaft 37 pushes the slant-cut teeth 38 into the proceeding notch 33 of the carrier 31, thus rotating the cassette shaft 37, and subsequently the attached cassette 10 by one notch. The number and geometry of slant-cut teeth 38 on the driving shaft 37, and carrier notches 33, control the resolution of indexing steps and ensure that the cartridges 1 within the cassette 10 are aligned axially with the plunger rod 42 of the drive unit 20.

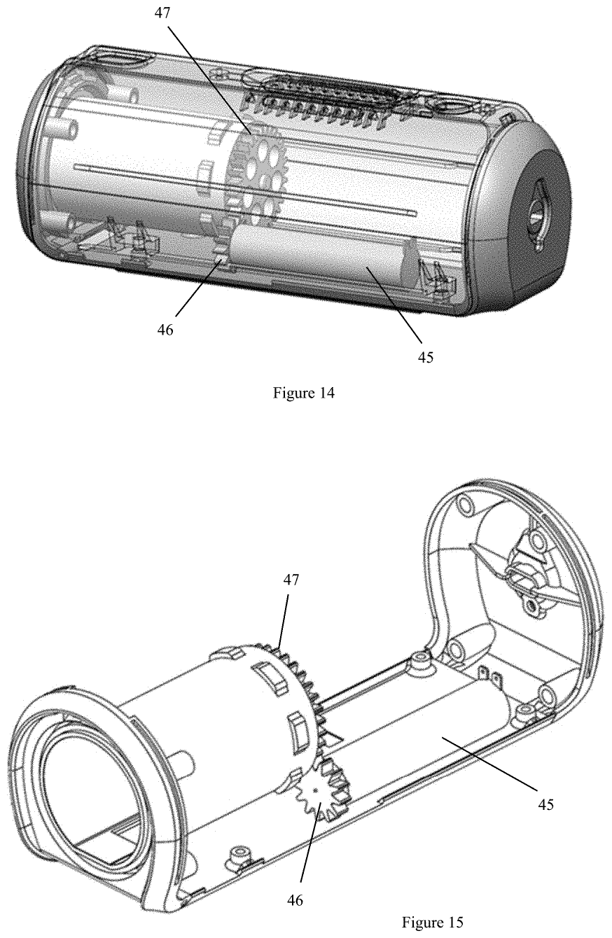

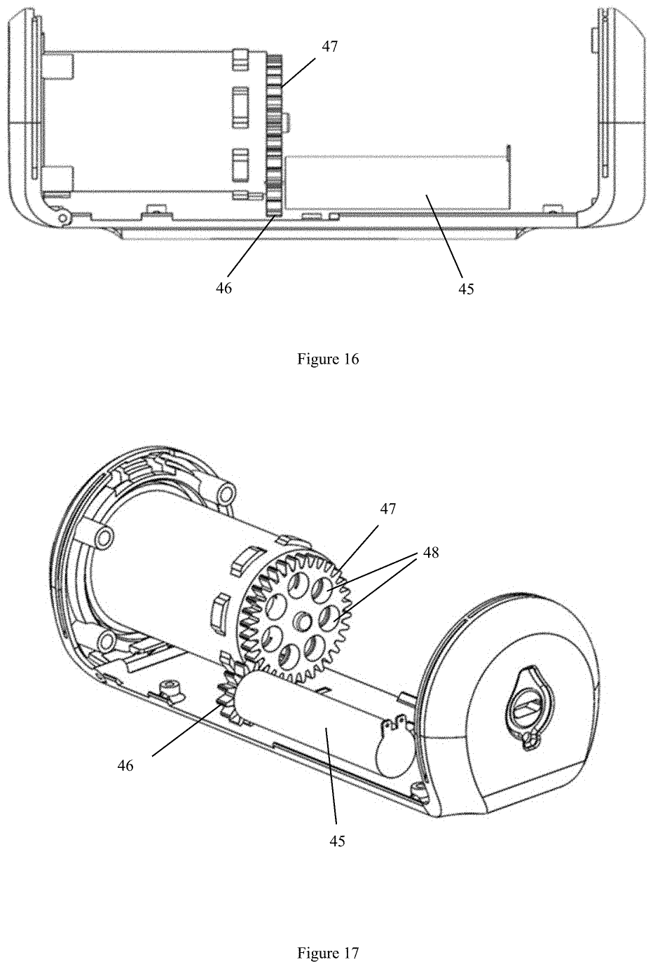

In alternative embodiments, as shown in FIGS. 14-17, alternative means of rotating and aligning the cassette 10 can be considered, e.g., a separate indexing drive motor 45 with a spur gear wheel 46 engaging with a toothed gear wheel 47 fabricated integrally on the rear of the retainer 30. With rotation of the spur gear wheel 46 by the drive motor 45, the gear wheel 47 is caused to rotate, along with the cassette 10. The drive motor 45 may be reversible allowing for bi-directional rotational adjustment of the cassette 10. The gear wheel 47 is provided with one or more apertures 48 through which the plunger rod 42 may pass to access an aligned cartridge 1. To dispense drug, the one or more apertures 48 are aligned with the plunger rod 42 in axial alignment with the aligned cartridge 1. Further arrangements are possible, such as ratchet and pawl type mechanisms.

Ramped surfaces 44 may be formed on the inner wall of the carrier 31, between the notches 33, to direct the slant-cut teeth 38 into the next notches 38 upon rotation. Preferably, the ramped surfaces 44 are ramped downwardly in the desired direction of rotation.

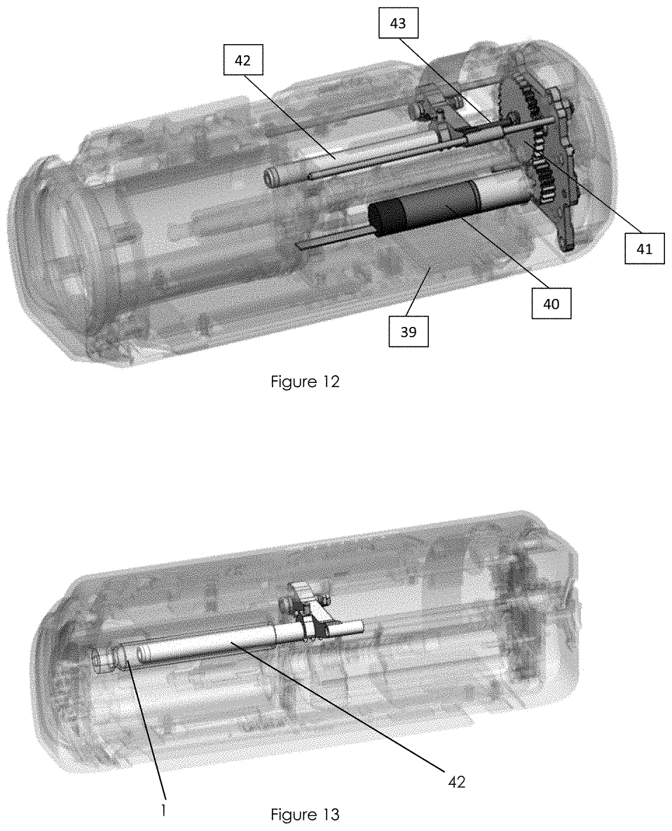

Internal components of the drive unit 20 are shown in FIG. 12. The main components of the infusion drive system are a battery 39, encoder motor 40, drivetrain 41, and plunger rod 42. During an infusion the encoder motor 40 is energized and will turn the drivetrain 41 to rotate the screw drive 43 to extend the plunger rod 42 forward from its home position and into the cassette drum 28. An encoder motor 40 and custom firmware are used in order to track the position of the plunger rod 42. The firmware also has the capability of monitoring the current of the encoder motor 40, which is directly correlated to the force that is being exerted by the plunger rod 42. As the plunger rod 42 enters the cassette drum 28 it passes through the cassette's main housing 5 via the plunger cutouts 8 under each cartridge 1. The revolver mechanism 29 ensures that the plunger rod 42 will be axially aligned with the cartridge 10. Upon further travel, the plunger rod 42 then enters into the cartridge 1 making contact with the cartridge stopper 3. The plunger rod 42 will continue to extend forward and will begin to drive the cartridge stopper 3 into the cartridge 1 (FIG. 13) expelling its contents into the cannula 13 piercing its septum 2, into the cassette top manifold 12, out into the infusion set 16, and into the patient. Once the contents of the cartridge 1 have been fully expelled, the encoder motor 40 will then be reversed to retract the plunger rod 42 back to its home position. Upon reaching home position, the plunger rod 42 will be retracted further past home position to interface with the driving shaft 34 of the revolver mechanism 29. Upon further retraction of the plunger rod 42 the driving shaft 34 of the revolver mechanism 29 is driven into the carrier 31 resulting in the indexing of cassette drum 28 by one notch, or cartridge position 1. Once the revolver index has completed the plunger rod 42 resets to its home position to repeat the process for the proceeding cartridge 1 in the cassette 10. In embodiments, the rigid plunger rod 42 could be replaced with a flexible or telescoping plunger rod. Likewise, the means of driving the plunger rod 42 could be substituted with a linear actuator, pneumatic, magnetic, or spring based system.

As will be appreciated by those skilled in the art, the subject invention may be used to dose the cartridges 1 in various sequences, including variations in full and partial dosing of the cartridges 1. For example, the cassette 10 may be caused to rotate to allow for delivery in sequential order of the cartridges 1 or one or more of the cartridges 1 may be skipped with additional rotation, allowing for later delivery. Also, the plunger rod 42 may be used to cause partial dosing of one or more of the cartridges 1, allowing for return to permit further delivery from the same cartridge 1, thus allowing for multiple dosings from one or more of the cartridges 1, in an established pattern. The flexibility in operation allows for multiple drugs to be delivered in different amounts and in different sequences, allowing for different combinations being delivered at different instances.

In one embodiment, any of the combinatorial drug delivery devices disclosed herein is able to deliver two or more drugs for the benefit of the patient suffering from any of a wide range of diseases or conditions, e.g., cancer, autoimmune disorder, inflammatory disorder, cardiovascular disease or fibrotic disorder. In one embodiment, one or more of the cartridges 1 may contain a single drug. In one embodiment, one or more of the cartridges 1 may contain two or more co-formulated drugs. In one embodiment, one or more of the cartridges 1 may contain a drug in solid form (such as a tablet, capsule, powder, lyophilized, spray dried), which can be reconstituted with flow of a diluent therein to form a liquid drug.

In one embodiment, one or more of the drugs of any of the combinatorial drug delivery devices disclosed herein is an immune checkpoint inhibitor. In certain embodiments, the immune checkpoint inhibitor is Programmed Death-1 (“PD-1”) pathway inhibitor, a cytotoxic T-lymphocyte-associated antigen 4 (“CTLA-4”) antagonist, a Lymphocyte Activation Gene-3 (“LAG3”) antagonist, a CD80 antagonist, a CD86 antagonist, a T cell immunoglobulin and mucin domain (“Tim-3”) antagonist, a T cell immunoreceptor with Ig and ITIM domains (“TIGIT”) antagonist, a CD20 antagonist, a CD96 antagonist, a Indoleamine 2,3-dioxygenase (“IDO1”) antagonist, a stimulator of interferon genes (“STING”) antagonist, a GARP antagonist, a CD40 antagonist, Adenosine A2A receptor (“A2aR”) antagonist, a CEACAM1 (CD66a) antagonist, a CEA antagonist, a CD47 antagonist, a Receptor Related Immunoglobulin Domain Containing Protein (“PVRIG”) antagonist, a tryptophan 2,3-dioxygenase (“TDO”) antagonist, a V-domain Ig suppressor of T cell activation (“VISTA”) antagonist, or a Killer-cell Immunoglobulin-like Receptor (“KIR”) antagonist.

In one embodiment, the PD-1 pathway inhibitor is an anti-PD-1 antibody or antigen binding fragment thereof. In certain embodiments, the anti-PD-1 antibody is pembrolizumab (KEYTRUDA; MK-3475), pidilizumab (CT-011), nivolumab (OPDIVO; BMS-936558), PDR001, MEDI0680 (AMP-514), TSR-042, REGN2810, JS001, AMP-224 (GSK-2661380), PF-06801591, BGB-A317, BI 754091, or SHR-1210.

In one embodiment, the PD-1 pathway inhibitor is an anti-PD-L1 antibody or antigen binding fragment thereof. In certain embodiments, the anti-PD-L1 antibody is atezolizumab (TECENTRIQ; RG7446; MPDL3280A; R05541267), durvalumab (MEDI4736), BMS-936559, avelumab (bavencio), LY3300054, CX-072 (Proclaim-CX-072), FAZ053, KN035, or MDX-1105.

In one embodiment, the PD-1 pathway inhibitor is a small molecule drug. In certain embodiments, the PD-1 pathway inhibitor is CA-170. In another embodiment, the PD-1 pathway inhibitor is a cell based therapy. In one embodiment, the cell based therapy is a MiHA-loaded PD-L1/L2-silenced dendritic cell vaccine. In other embodiments, the cell based therapy is an anti-programmed cell death protein 1 antibody expressing pluripotent killer T lymphocyte, an autologous PD-1-targeted chimeric switch receptor-modified T lymphocyte, or a PD-1 knockout autologous T lymphocyte.

In one embodiment, the PD-1 pathway inhibitor is an anti-PD-L2 antibody or antigen binding fragment thereof. In another embodiment, the anti-PD-L2 antibody is rHIgM12B7.

In one embodiment, the PD-1 pathway inhibitor is a soluble PD-1 polypeptide. In certain embodiments, the soluble PD-1 polypeptide is a fusion polypeptide. In some embodiments, the soluble PD-1 polypeptide comprises a ligand binding fragment of the PD-1 extracellular domain. In other embodiments, the soluble PD-1 polypeptide comprises a ligand binding fragment of the PD-1 extracellular domain. In another embodiment, the soluble PD-1 polypeptide further comprises an Fc domain.

In one embodiment, the immune checkpoint inhibitor is a CTLA-4 antagonist. In certain embodiments, the CTLA-4 antagonist is an anti-CTLA-4 antibody or antigen binding fragment thereof In some embodiments, the anti-CTLA-4 antibody is ipilimumab (YERVOY), tremelimumab (ticilimumab; CP-675,206), AGEN-1884, or ATOR-1015. In one embodiment, any of the combinatorial drug delivery devices disclosed herein includes a CTLA-4 antagonist, e.g., ipilimumab (YERVOY), and a PD-1 pathway inhibitor, e.g., nivolumab (OPDIVO) or pembrolizumab (KEYTRUDA).

In one embodiment, the immune checkpoint inhibitor is an antagonist of LAG3. In certain embodiments, the LAG3 antagonist is an anti-LAG3 antibody or antigen binding fragment thereof. In certain embodiments, the anti-LAG3 antibody is relatlimab (BMS-986016), MK-4280 (28G-10), REGN3767, GSK2831781, IMP731 (H5L7BW), BAP050, IMP-701 (LAG-5250), IMP321, TSR-033, LAG525, BI 754111, or FS-118. In one embodiment, any of the combinatorial drug delivery devices disclosed herein includes a LAG3 antagonist, e.g., relatlimab or MK-4280, and a PD-1 pathway inhibitor, e.g., nivolumab (OPDIVO) or pembrolizumab (KEYTRUDA). In one embodiment, any of the combinatorial drug delivery devices disclosed herein includes a LAG3 antagonist, e.g., relatlimab or MK-4280, and a CTLA-4 antagonist, e.g., ipilimumab (YERVOY). In one embodiment, any of the combinatorial drug delivery devices disclosed herein includes a LAG3 antagonist, e.g., relatlimab or MK-4280, a CTLA-4 antagonist, e.g., ipilimumab (YERVOY), and a PD-1 pathway inhibitor, e.g., nivolumab (OPDIVO) or pembrolizumab (KEYTRUDA).

In one embodiment, the immune checkpoint inhibitor is a KIR antagonist. In certain embodiments, the KIR antagonist is an anti-KIR antibody or antigen binding fragment thereof. In some embodiments, the anti-KIR antibody is lirilumab (1-7F9, BMS-986015, IPH 2101) or IPH4102.

In one embodiment, the immune checkpoint inhibitor is TIGIT antagonist. In one embodiment, the TIGIT antagonist is an anti-TIGIT antibody or antigen binding fragment thereof. In certain embodiments, the anti-TIGIT antibody is BMS-986207, AB 154, COM902 (CGEN-15137), or OMP-313M32.

In one embodiment, the immune checkpoint inhibitor is Tim-3 antagonist. In certain embodiments, the Tim-3 antagonist is an anti-Tim-3 antibody or antigen binding fragment thereof. In some embodiments, the anti-Tim-3 antibody is TSR-022 or LY3321367.

In one embodiment, the immune checkpoint inhibitor is an IDO1 antagonist. In another embodiment, the IDO1 antagonist is indoximod (NLG8189; 1-methyl-_{D}-TRP), epacadostat (INCB-024360, INCB-24360), KHK2455, PF-06840003, navoximod (RG6078, GDC-0919, NLG919), BMS-986205 (F001287), or pyrrolidine-2,5-dione derivatives.

In one embodiment, the immune checkpoint inhibitor is a STING antagonist. In certain embodiments, the STING antagonist is 2′ or 3′-mono-fluoro substituted cyclic-di-nucleotides; 2′3′-di-fluoro substituted mixed linkage 2′,5′-3′,5′ cyclic-di-nucleotides; 2′-fluoro substituted, bis-3′,5′ cyclic-di-nucleotides; 2′,2″-diF-Rp,Rp,bis-3′,5′ cyclic-di-nucleotides; or fluorinated cyclic-di-nucleotides.

In one embodiment, the immune checkpoint inhibitor is CD20 antagonist. In some embodiments, the CD20 antagonist is an anti-CD20 antibody or antigen binding fragment thereof. In one embodiment, the anti-CD20 antibody is rituximab (RITUXAN; IDEC-102; IDEC-C2B8), ABP 798, ofatumumab, or obinutuzumab.

In one embodiment, the immune checkpoint inhibitor is CD80 antagonist. In certain embodiments, the CD80 antagonist is an anti-CD80 antibody or antigen binding fragment thereof. In one embodiment, the anti-CD80 antibody is galiximab or AV 1142742.

In one embodiment, the immune checkpoint inhibitor is a GARP antagonist. In some embodiments, the GARP antagonist is an anti-GARP antibody or antigen binding fragment thereof. In certain embodiments, the anti-GARP antibody is ARGX-115.

In one embodiment, the immune checkpoint inhibitor is a CD40 antagonist. In certain embodiments, the CD40 antagonist is an anti-CD40 antibody for antigen binding fragment thereof. In some embodiments, the anti-CD40 antibody is BMS3h-56, lucatumumab (HCD122 and CHIR-12.12), CHIR-5.9, or dacetuzumab (huS2C6, PRO 64553, RG 3636, SGN 14, SGN-40). In another embodiment, the CD40 antagonist is a soluble CD40 ligand (CD40L). In one embodiment, the soluble CD40 ligand is a fusion polypeptide. In one embodiment, the soluble CD40 ligand is a CD40L/FC2 or a monomeric CD40-L.

In one embodiment, the immune checkpoint inhibitor is an A2aR antagonist. In some embodiments, the A2aR antagonist is a small molecule. In certain embodiments, the A2aR antagonist is CPI-444, PBF-509, istradefylline (KW-6002), preladenant (SCH420814), tozadenant (SYN115), vipadenant (BIIB014), HTL-1071, ST1535, SCH412348, SCH442416, SCH58261, ZM241385, or AZD4635.

In one embodiment, the immune checkpoint inhibitor is a CEACAM1 antagonist. In some embodiments, the CEACAM1 antagonist is an anti-CEACAM1 antibody or antigen binding fragment thereof. In one embodiment, the anti-CEACAM1 antibody is CM-24 (MK-6018).

In one embodiment, the immune checkpoint inhibitor is a CEA antagonist. In one embodiment, the CEA antagonist is an anti-CEA antibody or antigen binding fragment thereof. In certain embodiments, the anti-CEA antibody is cergutuzumab amunaleukin (RG7813, RO-6895882) or RG7802 (RO6958688).

In one embodiment, the immune checkpoint inhibitor is a CD47 antagonist. In some embodiments, the CD47 antagonist is an anti-CD47 antibody or antigen binding fragment thereof. In certain embodiments, the anti-CD47 antibody is HuF9-G4, CC-90002, TTI-621, ALX148, NI-1701, NI-1801, SRF231, or Effi-DEM.

In one embodiment, the immune checkpoint inhibitor is a PVRIG antagonist. In certain embodiments, the PVRIG antagonist is an anti-PVRIG antibody or antigen binding fragment thereof. In one embodiment, the anti-PVRIG antibody is COM701 (CGEN-15029).

In one embodiment, the immune checkpoint inhibitor is a TDO antagonist. In one embodiment, the TDO antagonist is a 4-(indol-3-yl)-pyrazole derivative, a 3-indol substituted derivative, or a 3-(indol-3-yl)-pyridine derivative. In another embodiment, the immune checkpoint inhibitor is a dual IDO and TDO antagonist. In one embodiment, the dual IDO and TDO antagonist is a small molecule.

In one embodiment, the immune checkpoint inhibitor is a VISTA antagonist. In some embodiments, the VISTA antagonist is CA-170 or JNJ-61610588.

In one embodiment, one or more of the drugs of any of the combinatorial drug delivery devices disclosed herein is an immune checkpoint enhancer or stimulator.

In one embodiment, the immune checkpoint enhancer or stimulator is a CD28 agonist, a 4-1BB agonist, an OX40 agonist, a CD27 agonist, a CD80 agonist, a CD86 agonist, a CD40 agonist, an ICOS agonist, a CD70 agonist, or a GITR agonist.

In one embodiment, the immune checkpoint enhancer or stimulator is an OX40 agonist. In certain embodiments, the OX40 agonist is an anti-OX40 antibody or antigen binding fragment thereof. In some embodiments, the anti-OX40 antibody is tavolixizumab (MEDI-0562), pogalizumab (MOXR0916, RG7888), GSK3174998, ATOR-1015, MEDI-6383, MEDI-6469, BMS 986178, PF-04518600, or RG7888 (MOXR0916). In another embodiment, the OX40 agonist is a cell based therapy. In certain embodiments, the OX40 agonist is a GINAKIT cell (iC9-GD2-CD28-OX40-expressing T lymphocytes).

In one embodiment, the immune checkpoint enhancer or stimulator is a CD40 agonist. In some embodiments, the CD40 agonist is an anti-CD40 antibody or antigen binding fragment thereof. In one embodiment, the anti-CD40 antibody is ADC-1013 (JNJ-64457107), RG7876 (RO-7009789), HuCD40-M2, APX005M (EPI-0050), or Chi Lob 7/4. In another embodiment, the CD40 agonist is a soluble CD40 ligand (CD40-L). In one embodiment, the soluble CD40 ligand is a fusion polypeptide. In certain embodiments, the soluble CD40 ligand is a trimeric CD40-L (AVREND®).

In one embodiment, the immune checkpoint enhancer or stimulator is a GITR agonist. In certain embodiments, the GITR agonist is an anti-GITR antibody or antigen binding fragment thereof. In one embodiment, the anti-GITR antibody is BMS-986156, TRX518, GWN323, INCAGN01876, or MEDI1873. In one embodiment, the GITR agonist is a soluble GITR ligand (GITRL). In some embodiments, the soluble GITR ligand is a fusion polypeptide. In another embodiment, the GITR agonist is a cell based therapy. In one embodiment, the cell based therapy is an anti-CTLA4 mAb RNA/GITRL RNA-transfected autologous dendritic cell vaccine or a GITRL RNA-transfected autologous dendritic cell vaccine.

In one embodiment, the immune checkpoint enhancer or stimulator a 4-1BB agonist. In some embodiments, the 4-1BB agonist is an anti-4-1BB antibody or antigen binding fragment thereof. In one embodiment, the anti-4-1BB antibody is urelumab or PF-05082566.

In one embodiment, the immune checkpoint enhancer or stimulator is a CD80 agonist or a CD86 agonist. In some embodiments, the CD80 agonist or the CD86 agonist is a soluble CD80 or CD86 ligand (CTLA-4). In certain embodiments, the soluble CD80 or CD86 ligand is a fusion polypeptide. In one embodiment, the CD80 or CD86 ligand is CTLA4-Ig (CTLA4-IgG4m, RG2077, or RG1046) or abatacept (ORENCIA, BMS-188667). In other embodiments, the CD80 agonist or the CD86 agonist is a cell based therapy. In one embodiment, the cell based therapy is MGN1601 (an allogeneic renal cell carcinoma vaccine).

In one embodiment, the immune checkpoint enhancer or stimulator is a CD28 agonist. In some embodiments, the CD28 agonist is an anti-CD28 antibody or antigen binding fragment thereof. In certain embodiments, the anti-CD28 antibody is TGN1412.

In one embodiment, the CD28 agonist is a cell based therapy. In certain embodiments, the cell based therapy is JCAR015 (anti-CD19-CD28-zeta modified CAR CD3+ T lymphocyte); CD28CAR/CD137CAR-expressing T lymphocyte; allogeneic CD4+ memory Th1-like T cells/microparticle-bound anti-CD3/anti-CD28; anti-CD19/CD28/CD3zeta CAR gammaretroviral vector-transduced autologous T lymphocytes KTE-C19; anti-CEA IgCD28TCR-transduced autologous T lymphocytes; anti-EGFRvIII CAR-transduced allogeneic T lymphocytes; autologous CD123CAR-CD28-CD3zeta-EGFRt-expressing T lymphocytes; autologous CD171-specific CAR-CD28 zeta-4-1-BB-EGFRt-expressing T lymphocytes; autologous CD19CAR-CD28-CD3zeta-EGFRt-expressing Tcm-enriched T cells; autologous PD-1-targeted chimeric switch receptor-modified T lymphocytes (chimera with CD28); CD19CAR-CD28-CD3zeta-EGFRt-expressing Tcm-enriched T lymphocytes; CD19CAR-CD28-CD3zeta-EGFRt-expressing Tn/mem-enriched T lymphocytes; CD19CAR-CD28zeta-4-1BB-expressing allogeneic T lymphocytes; CD19CAR-CD3zeta-4-1BB-CD28-expressing autologous T lymphocytes; CD28CAR/CD137CAR-expressing T lymphocytes; CD3/CD28 costimulated vaccine-primed autologous T lymphocytes; or iC9-GD2-CD28-OX40-expressing T lymphocytes.

In one embodiment, the immune checkpoint enhancer or stimulator is a CD27 agonist. In certain embodiments, the CD27 agonist is an anti-CD27 antibody or antigen binding fragment thereof. In one embodiment, the anti-CD27 antibody is varlilumab (CDX-1127).

In one embodiment, the immune checkpoint enhancer or stimulator is a CD70 agonist. In some embodiments, the CD70 agonist is an anti-CD70 antibody or antigen binding fragment thereof. In one embodiment, the anti-CD70 antibody is ARGX-110.

In one embodiment, the immune checkpoint enhancer or stimulator is an ICOS agonist. In certain embodiments, the ICOS agonist is an anti-ICOS antibody or antigen binding fragment thereof. In some embodiments, the anti-ICOS antibody is BMS986226, MEDI-570, GSK3359609, or JTX-2011. In other embodiments, the ICOS agonist is a soluble ICOS ligand. In some embodiments, the soluble ICOS ligand is a fusion polypeptide. In one embodiment, the soluble ICOS ligand is AMG 750.

In one embodiment, one or more of the drugs of any of the combinatorial drug delivery devices disclosed herein is an anti-CD73 antibody or antigen binding fragment thereof. In certain embodiments, the anti-CD73 antibody is MEDI9447.

In one embodiment, one or more of the drugs of any of the combinatorial drug delivery devices disclosed herein is a TLR9 agonist. In one embodiment, the TLR9 agonist is agatolimod sodium.

In one embodiment, one or more of the drugs of any of the combinatorial drug delivery devices disclosed herein is a cytokine. In certain embodiments, the cytokine is a chemokine, an interferon, an interleukin, lymphokine, or a member of the tumor necrosis factor family. In some embodiments, the cytokine is IL-2, IL-15, or interferon-gamma.

In one embodiment, one or more of the drugs of any of the combinatorial drug delivery devices disclosed herein is a TGF-β antagonist. In some embodiments, the TGF-β antagonist is fresolimumab (GC-1008); NIS793; IMC-TR1 (LY3022859); ISTH0036; trabedersen (AP 12009); recombinant transforming growth factor-beta-2; autologous HPV-16/18 E6/E7-specific TGF-beta-resistant T lymphocytes; or TGF-beta-resistant LMP-specific cytotoxic T-lymphocytes.

In one embodiment, one or more of the drugs of any of the combinatorial drug delivery devices disclosed herein is an iNOS antagonist. In some embodiments, the iNOS antagonist is N-Acetyle-cysteine (NAC), aminoguanidine, L-nitroarginine methyl ester, or S,S-1,4-phenylene-bis(1,2-ethanediyl)bis-isothiourea).

In one embodiment, one or more of the drugs of any of the combinatorial drug delivery devices disclosed herein is a SHP-1 antagonist.

In one embodiment, one or more of the drugs of any of the combinatorial drug delivery devices disclosed herein is a colony stimulating factor 1 receptor (“CSF1R”) antagonist. In certain embodiments, the CSF1R antagonist is an anti-CSF1R antibody or antigen binding fragment thereof. In some embodiments, the anti-CSF1R antibody is emactuzumab.

In one embodiment, one or more of the drugs of any of the combinatorial drug delivery devices disclosed herein is an agonist of a TNF family member. In some embodiments, the agonist of the TNF family member is ATOR 1016, ABBV-621, or Adalimumab.

In one embodiment, one or more of the drugs of any of the combinatorial drug delivery devices disclosed herein is an Interleukin-2 (IL-2), such as aldesleukin. Preferably, the IL-2 or conjugated IL-2 (e.g., pegylated) has been modified to selectively activate T-effector cells over T-regulatory cells (“T-eff IL-2”), such as bempegaldesleukin. In one embodiment, any of the combinatorial drug delivery devices disclosed herein includes a modified IL-2, such as bempegaldesleukin, which selectively activates T-effector cells over T-regulatory cells, and a PD-1 pathway inhibitor, e.g., nivolumab (OPDIVO) or pembrolizumab (KEYTRUDA). In one embodiment, any of the combinatorial drug delivery devices disclosed herein includes a modified IL-2, such as bempegaldesleukin, which selectively activates T-effector cells over T-regulatory cells, and a LAG3 antagonist, e.g., relatlimab or MK-4280. In one embodiment, any of the combinatorial drug delivery devices disclosed herein includes a modified IL-2, such as bempegaldesleukin, which selectively activates T-effector cells over T-regulatory cells, and a PD-1 pathway inhibitor, e.g., nivolumab (OPDIVO) or pembrolizumab (KEYTRUDA), and a LAG3 antagonist, e.g., relatlimab or MK-4280. In one embodiment, any of the combinatorial drug delivery devices disclosed herein includes a modified IL-2, such as bempegaldesleukin, which selectively activates T-effector cells over T-regulatory cells and a CTLA-4 antagonist, e.g., ipilimumab (YERVOY). In one embodiment, any of the combinatorial drug delivery devices disclosed herein includes a modified IL-2, such as bempegaldesleukin, which selectively activates T-effector cells over T-regulatory cells, a PD-1 pathway inhibitor, e.g., nivolumab (OPDIVO) or pembrolizumab (KEYTRUDA), and a CTLA-4 antagonist, e.g., ipilimumab (YERVOY). In one embodiment, any of the combinatorial drug delivery devices disclosed herein includes a modified IL-2, such as bempegaldesleukin, which selectively activates T-effector cells over T-regulatory cells, a CTLA-4 antagonist, e.g., ipilimumab (YERVOY), and a LAG3 antagonist, e.g., relatlimab or MK-4280. In one embodiment, any of the combinatorial drug delivery devices disclosed herein includes a modified IL-2, such as bempegaldesleukin, which selectively activates T-effector cells over T-regulatory cells, a PD-1 pathway inhibitor, e.g., nivolumab (OPDIVO) or pembrolizumab (KEYTRUDA), a CTLA-4 antagonist, e.g., ipilimumab (YERVOY), and a LAG3 antagonist, e.g., relatlimab or MK-4280.

In one embodiment, one or more of the drugs of any of the combinatorial drug delivery devices disclosed herein is a CD160 (NK1) agonist. In certain embodiments, the CD160 (NK1) agonist is an anti-CD160 antibody or antigen binding fragment thereof. In one embodiment, the anti-CD160 antibody is BY55.

In one embodiment, the one or more of the cartridges 1 may contain a soluble CTLA-4 polypeptide, which can be useful for treating, for instance, T-cell mediated autoimmune disorders, such as rheumatoid arthritis, juvenile idiopathic arthritis, psoriatic arthritis, graft-versus-host disease, and transplant rejection. In one embodiment, the soluble CTLA-4 polypeptide is abatacept (ORENCIA), belatacept (NULOJIX), RG2077, or RG-1046. In certain embodiments, one or more of the cartridges 1 of a combinatorial drug delivery device as described herein include a soluble CTLA-4 polypeptide, e.g., abatacept (ORENCIA) and a Bruton's tyrosine kinase inhibitor, e.g., branebrutinib. In certain embodiments, one or more of the cartridges 1 of a combinatorial drug delivery device as described herein include a soluble CTLA-4 polypeptide, e.g., abatacept (ORENCIA) and a tyrosine kinase-2 inhibitor, e.g., BMS-986165. In certain embodiments, one or more of the cartridges 1 of a combinatorial drug delivery device as described herein include a soluble CTLA-4 polypeptide, e.g., abatacept (ORENCIA) and an Interleukin-2 (IL-2) or “T-reg IL-2”, which selectively activates T-regulatory cells as opposed to T-effector cells, e.g., BMS-986326 and NKTR-358.

What is claimed is:

1. A drug delivery device for delivering drug from a plurality of drug cartridges to a patient, each of the drug cartridges including an elongated body having a first end sealed with a septum and a second open end, and, a stopper located in the body, wherein, in an initial state, each of the drug cartridges includes at least one drug contained in the body between the stopper and the septum thereof, the drug delivery device comprising:
a cylindrical cassette configured to accommodate the plurality of drug cartridges;
a plurality of cannulas positioned to simultaneously pierce the septa of the plurality of drug cartridges;
a plurality of fluidic channels individually connected to the plurality of cannulas, the plurality of fluidic channels converging to a common outlet;
a reversibly advanceable plunger; and,
an indexer for incrementally rotating the cassette to align the plurality of drug cartridges individually with the plunger, the plunger being advanceable to urge the stopper of the aligned drug cartridge towards the septum of the aligned drug cartridge to cause the at least one drug contained in the body of the aligned drug cartridge to be expelled through the cannula piercing the septum of the aligned drug cartridge.

2. (canceled)

3. A drug delivery device as in claim 11, wherein the indexer further includes a tubular carrier, the first and second shafts extending into the carrier.

4. A drug delivery device as in claim 3, wherein the carrier includes a plurality of notches defined on an inner surface thereof, wherein the first shaft includes a plurality of slant-cut teeth formed to interface with the plurality of notches, and, wherein the first shaft is biased to have the slant-cut teeth normally interface with the plurality of notches.

5. A drug delivery device as in claim 4, wherein the second shaft includes a plurality of teeth, wherein, with the axial shifting of the second shaft relative to the first shaft, the plurality of teeth engage the plurality of slant-cut teeth to cause the plurality of slant-cut teeth to separate from the plurality of notches.

6. A drug delivery device as in claim 5, wherein, with return of the second shaft to being spaced from the first shaft, the plurality of slant-cut teeth are incrementally rotated and urged into engagement with the plurality of notches.

7. A drug delivery device as in claim 4, wherein the carrier includes a plurality of ramped surfaces defined on the inner surface thereof, the ramped surfaces being located among the plurality notches.

8. A drug delivery device for delivering drug from a plurality of drug cartridges to a patient, each of the drug cartridges including an elongated body having a first end sealed with a septum and a second open end, and, a stopper located in the body, wherein, in an initial state, each of the drug cartridges includes at least one drug contained in the body between the stopper and the septum thereof, the drug delivery device comprising:
a cylindrical cassette configured to accommodate the plurality of drug cartridges;
a plurality of cannulas positioned to simultaneously pierce the septa of the plurality of drug cartridges;
a plurality of fluidic channels individually connected to the plurality of cannulas, the plurality of fluidic channels converging to a common outlet for delivery to the patient;
a reversibly advanceable plunger;
an indexing drive motor;
a spur gear wheel coupled to the indexing drive motor; and,
a toothed gear wheel coupled to the cassette, and in engagement with the spur gear wheel, wherein, with rotation of the spur gear wheel by the indexing drive motor, the toothed gear wheel is caused to rotate along with the cassette to align the plurality of drug cartridges individually with the plunger, the plunger being advanceable to urge the stopper of the aligned drug cartridge towards the septum of the aligned drug cartridge to cause the at least one drug contained in the body of the aligned drug cartridge to be expelled through the cannula piercing the septum of the aligned drug cartridge.

9. A drug delivery device as in claim 8, wherein, the indexing drive motor is reversible.

10. A drug delivery device as in claim 8, wherein, one or more apertures are formed in the toothed gear wheel, and, wherein the plunger accesses the aligned drug cartridge through the one or more apertures.

11. A drug delivery device as in claim 1, wherein the indexer including:
a first shaft coupled to the cassette so as to be rotatable therewith; and,
a second shaft coaxially aligned with, and normally biased to be spaced from, the first shaft,
wherein, the second shaft is axially shiftable to engage the first shaft, and, wherein, the first and second shafts include cooperating elements which cause incremental rotation of the first shaft, relative to the second shaft, upon the second shaft engaging the first shaft.
